(19) United States
(12) Patent Application Publication
(10) Pub. No.: US 2014/0343461 A1
(43) Pub. Date: Nov. 20, 2014

(54) LIGAMENT LAXITY MEASURING SYSTEM AND METHOD FOR MEASURING LIGMENT LAXITY

(71) Applicant: National Taiwan Ocean University, Keelung (TW)

(72) Inventor: Chen-Chou LIN, Taipei City (TW)

(73) Assignee: National Taiwan Ocean University, Keelung (TW)

(21) Appl. No.: 14/279,850

(22) Filed: May 16, 2014

(30) Foreign Application Priority Data
May 17, 2013 (TW) 102117610

(51) Int. Cl. A61B 5/11; A61B 5/22

(52) U.S. Cl. CPC A61B 5/1122; A61B 5/22
USPC 600/595

(57) ABSTRACT

A ligament laxity measuring system for measuring ligament laxity between a first limb and a second limb connected to each other includes a force module, an angle sensing module and a data processing module. The force module disposed on the first limb is adapted to provide an external force to make the first limb pivot or twist with respect to the second limb, thereby generating a pivoting (varus/valgus) value and a twisting (rotational) value. The force module generates a force signal based on the external force. Opposite two segments of the angle sensing module are disposed on the first limb and the second limb respectively. The angle sensing module is used for sensing the pivoting value and the twisting value of the first limb and generating an angle signal. The data processing module is connected to the force sensor of the force module and the angle sensing module.

### CROSS-REFERENCE TO RELATED APPLICATIONS

This non-provisional application claims priority under 35 U.S.C. §119(a) on Patent Application No(s). 102117610 filed in Taiwan, R.O.C. on May 17, 2013, the entire contents of which are hereby incorporated by reference.

### TECHNICAL FIELD

The disclosure relates to a measuring system, more particularly to a ligament laxity measuring system.

### BACKGROUND

Knee ligament injuries or knee ligament rupture are common sports injures. The treatment for these injures are applied based on patients' background or conditions, such as ages, athletic ability, stability of the knee and other injures involved.

During diagnoses, doctors measure knee ligament laxity to determine the degree of injury of the knee. Measuring knee ligament laxity properly can provide vital information regarding patients' conditions for doctors to make decisions.

Nowadays the ligament laxity measuring equipments are able to measure knee ligament laxity of each part of the knee (e.g., the anterior cruciate ligament, the posterior cruciate ligament, the medial collateral ligament, the lateral collateral ligament). However, the design of the equipments is complicated and is hard to use so the patient needs to change postures to measure knee ligament laxity of each part of the knee, which is inconvenient for both doctors and patients.

Moreover, a traditional ligament laxity measuring equipment can measure linear displacements of joints (e.g. anterior or posterior) but it cannot measure relative angle between the limbs. In other words, the traditional ligament laxity measuring equipment is unable to provide enough information for doctors to determine the injury condition of the ligaments.

Additionally, it requires multiple measurements to obtain data of ligament laxity and this leads to the issue of deviation. Hence, in order to measure ligament laxity more precisely, it is important to develop a new ligament laxity measuring equipment capable of simplifying the measuring processes and providing the function of rotational displacements measurement.

### SUMMARY

A ligament laxity measuring system configured for measuring ligament laxity between a first limb and a second limb connected to each other comprises a force module, an angle sensing module and a data processing module. The force module is configured for being disposed on the first limb and for providing an external force to make the first limb pivot or twist with respect to the second limb, thereby generating a pivoting (varus/valgus) value and a twisting (rotational) value. The force module generates a force signal based on the external force. Opposite two segments of the angle sensing module are disposed on the first limb and the second limb respectively. The angle sensing module is configured for sensing the pivoting (varus/valgus) value and the twisting (rotational) value of the first limb and generating an angle signal. The data processing module is connected to the force sensor of the force module and the angle sensing module via data cables. The data processing module is configured for calculating a functional relationship of force and angle based on the force signal and the angle signal, and the ligament laxity is measured based on the functional relationship of force and angle.

A method for measuring ligament laxity, configured to measure ligament laxity between a first limb and a second limb connected to each other, comprises the steps of: enforcing an external force to a force module to make the first limb rotate with respect to the second limb, therefore generating a pivoting (varus/valgus) value to make a force sensor of the force module generate a force signal based on the external force, and to make an angle sensing module generate an angle signal based on the pivoting (varus/valgus) value at the same time; making a data processing module calculate a functional relationship of force and angle based on the force signal and the angle signal, for determining ligament laxity.

A method for measuring ligament laxity, configured to measure ligament laxity between a first limb and a second limb connected to each other, comprises the steps of: enforcing an external force to a force module to make the first limb rotate with respect to the second limb, therefore generating a twisting (rotational) value to make a force sensor of the force module generate a force signal based on the external force, and to make an angle sensing module generate an angle signal based on the twisting (rotational) value at the same time; making a data processing module calculate a functional relationship of force and angle based on the force signal and the angle signal, for determining ligament laxity.

### BRIEF DESCRIPTION OF THE DRAWINGS

The present disclosure will become more fully understood from the detailed description and the drawings given herein below for illustration only, and thus does not limit the present disclosure, wherein:

FIG. 1A is a perspective view of a ligament laxity measuring system according to a first embodiment of the disclosure;

FIG. 1B is a perspective view of a ligament laxity measuring system according to a second embodiment of the disclosure;

FIG. 2 is a perspective view of a force module of FIG. 1A;

FIG. 3 is a perspective view of a fixing module of FIG. 1A;

FIG. 4 is a flow chart of the process of the measuring method according to the first embodiment of the disclosure; and

FIG. 5 is a flow chart of the process of the measuring method according to the second embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

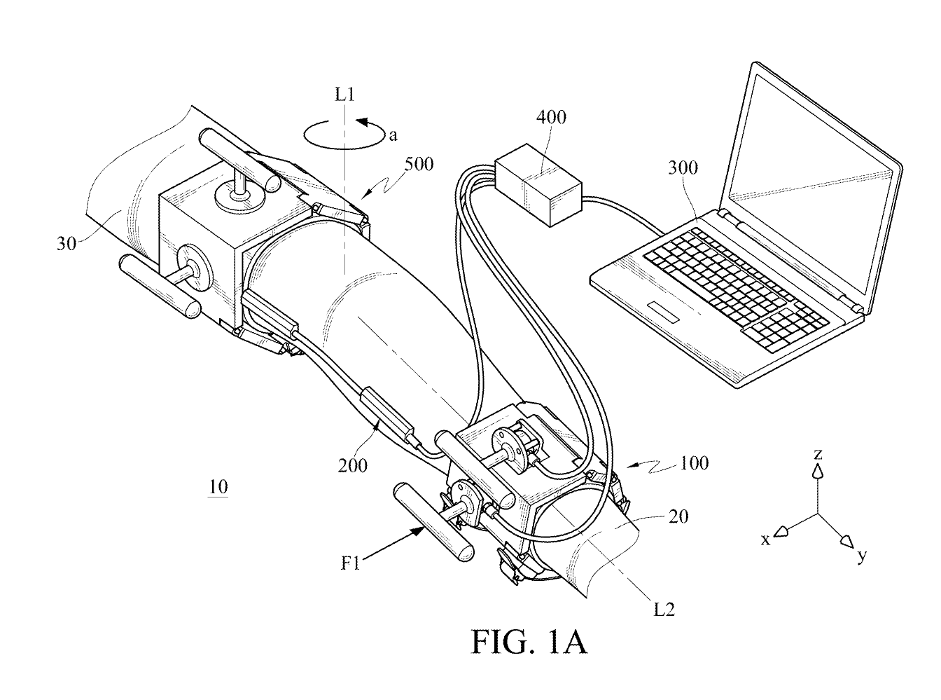

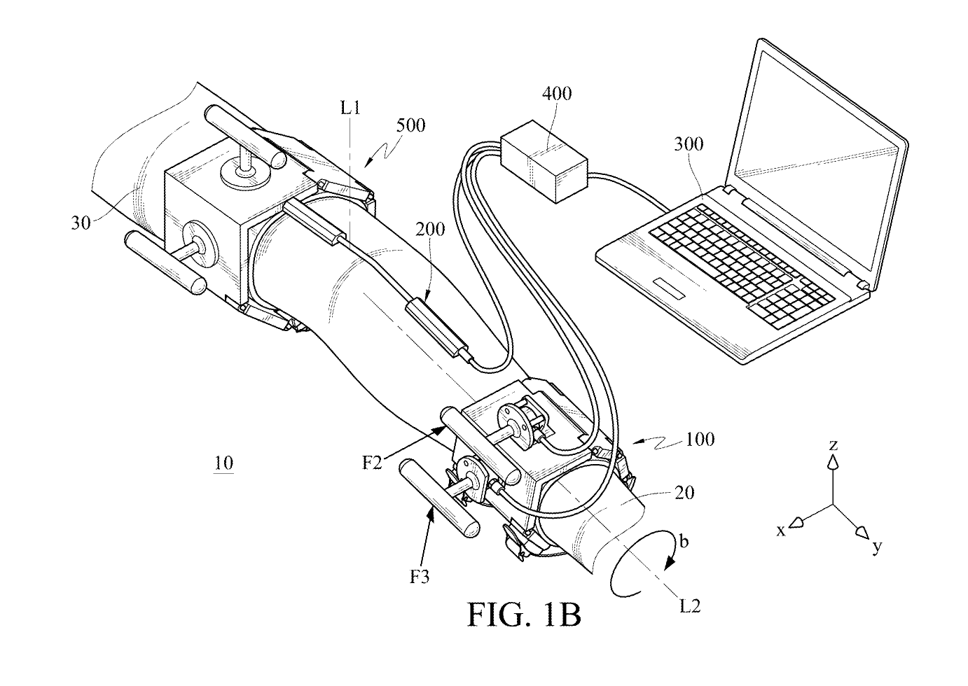

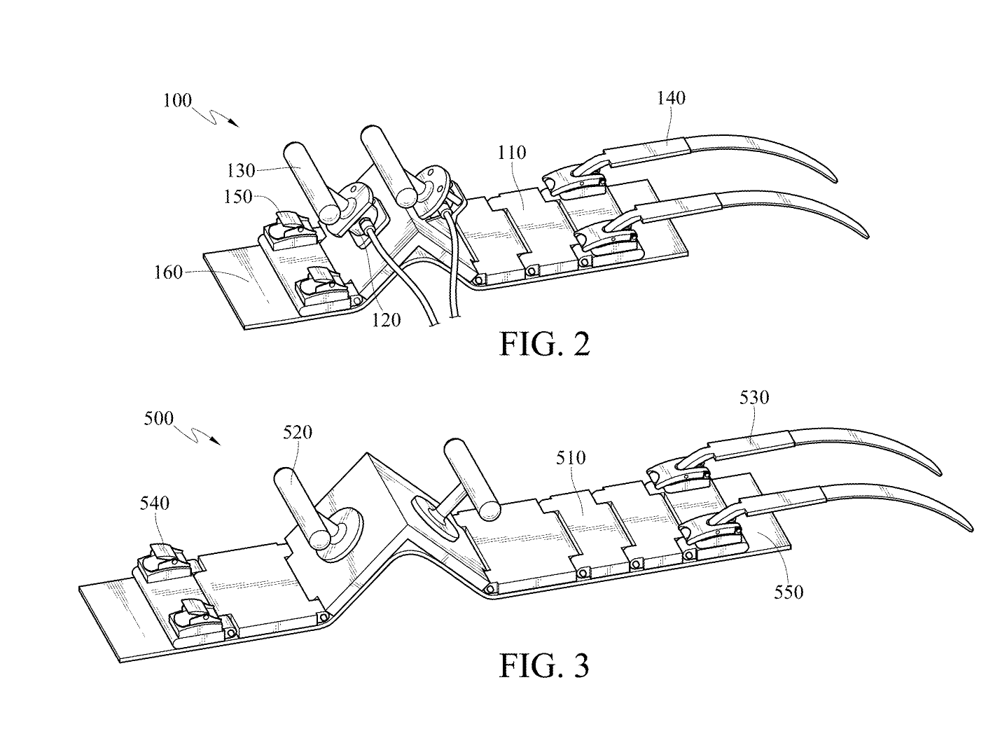

FIG. 1A is a perspective view of a ligament laxity measuring system according to a first embodiment of the disclosure; FIG. 1B is a perspective view of a ligament laxity measuring system according to a second embodiment of the disclosure; FIG. 2 is a perspective view of a force module of FIG. 1A; FIG. 3 is a perspective view of a fixing module of FIG. 1A. As seen in FIG. 1A to FIG. 3, the ligament laxity measuring system 10 of this embodiment is for measuring ligament laxity between a first limb 20 and a second limb 30. In this embodiment, the first limb 20 is a calf while the second limb 30 is a thigh, for example. Though the ligament laxity measuring system 10 of this embodiment is for measuring ligament laxity, it is not limited thereto. It may be used for doctors to diagnose injuries or to heal of the ligament.

In this embodiment, the ligament laxity measuring system 10 comprises a force module 100, an angle sensing module 200 and a data processing module 300. In addition, the ligament laxity measuring system 10 may further comprise a data collection module 400 and a fixing module 500.

The force module 100 comprises a first strap 110, a force sensor 120, at least one handle 130, a first fastener 140 and a second fastener 150. The force sensor 120 is disposed on the first strap 110. The handle 130 is connected to the force sensor 120. The first fastener 140 and the second fastener 150 are disposed on the opposite two ends of the first strap 110 respectively. The first fastener 140 is detachably fastened with the second fastener 150 to make the first strap 110 be sleeved on the first limb 20 in a detachable manner. In this embodiment, the force sensor 120 is a load cell (e.g. FUTEK LCM300). Furthermore, the number of the handles 130 of this embodiment is two. One of the handles 130 is located on the front side of the calf while the other one is located on the lateral side of the calf. Moreover, a silicone pad 160 may be disposed inside the first strap 110 to make users feel more comfortable.

The force module 100 is configured for providing an external force (e.g. a force generated by pushing) to make the first limb 20 pivot or twist with respect to the second limb 30, thereby generating a pivoting (varus/valgus) value or a twisting (rotational) value. The pivoting (varus/valgus) value or the twisting (rotational) value will be further explained later.

The force sensor 120 generates a force signal based on the external force.

In this embodiment, the force module 100 is sleeved on the first limb 20 by the straps and the fasteners, which is different from the traditional equipments which involve measuring sizes of the legs to manufacture the corresponding jigs. Thus, the force module 100 of this embodiment is capable of matching first limbs 20 of different sizes.

The opposite two segments of the angle sensing module 200 are configured for being disposed on the first limb 20 and the second limb 30 respectively. In this embodiment, the angle sensing module 200 is a strain gauge (e.g. Biometrics Ltd SG150 or Biometrics Ltd Q150). The opposite two segments of the angle sensing module 200 may be attached to the front sides of the first limb 20 and the second limb 30 respectively (as shown in FIG. 1A) or be attached to the lateral sides of the first limb 20 and the second limb 30 respectively (as shown in FIG. 1B).

The angle sensing module 200 is configured for detecting the pivoting (varus/valgus) value or the twisting (rotational) value of the first limb 20 and generates a corresponding angle signal. In this embodiment, the angle signal is a voltage signal. Referring to Table 1, adjusting the relationship between the output voltage and the pivoting (varus/valgus) values as well as the twisting (rotational) values of the first limb 20 and the second limb 30 before measuring ligament laxity can make the angle sensing module 200 generate the corresponding angle signal based on the pivoting (varus/valgus) value or the twisting (rotational) value. On the other hand, the pivoting (varus/valgus) value and the twisting (rotational) value can be derived from the angle signal of the angle sensing module 200.

TABLE 1
Relationships between Twisting (rotational) Values and
Output Voltage Differences and between Pivoting (varus/valgus)
Values and Output Voltage Differences

| twisting | | pivoting | |
|---|---|---|---|
| (rotational) | output voltage | (varus/valgus) | output voltage |
| value between | difference (mV) | value between | difference (mV) |
| first limb | of angle sensing | first limb | of angle sensing |
| and second | module | and second | module |
| limb (deg) | (Q150) | limb (deg) | (SG150) |
| 0 | 1.456554 | 0 | 1.011595 |
| 10 | 1.889828 | 10 | 1.491222 |
| 20 | 2.203579 | 20 | 1.959960 |
| 30 | 2.589177 | 30 | 2.502668 |
| 40 | 2.967565 | 40 | 3.048876 |
| 50 | 3.314534 | 50 | 3.541878 |

The data processing module 300 is connected to the force sensor 120 and the angle sensing module 200 via data cable. In this embodiment, the data processing module 300 is a notebook, but it is not limited thereto. In other embodiments, the data processing module 300 may be a desktop or a tablet computer.

The data processing module 300 is configured for calculating a functional relationship of force and angle based on the force signal and the angle signal and for further drawing a diagram of force and angle. The diagram of force and angle is a basis for judgment regarding ligament laxity.

The data collection module 400 is connected to the force sensor 120 and the data processing module 300 via data cable and is between them. Further, the data collection module 400 is connected to and is between the angle sensing module 200 and the data processing module 300 via data cable. In other words, the force sensor 120 and the angle sensing module 200 are connected via the data collection module 400 and the data processing module 300. In this embodiment, the data collection module 400 comprises a data collection box (e.g. InstruNet Network Device INET-100) and a collection controller (e.g. InstruNet Model i240 USB 2.0) which are not shown in the figures.

The fixing module 500 comprises a second strap 510, a fixing handle 520, a third fastener 530 and a fourth fastener 540. The fixing handle 520 is connected to the second strap 510. The third fastener 530 and the fourth fastener 540 are disposed on the opposite two ends of the second strap 510. The third fastener 530 is fastened with the fourth fastener 540 in a detachable way to make the second strap 510 be detachably sleeved on the second limb 30. Moreover, a silicone pad 550 may be disposed inside the second strap 510 to make users feel more comfortable.

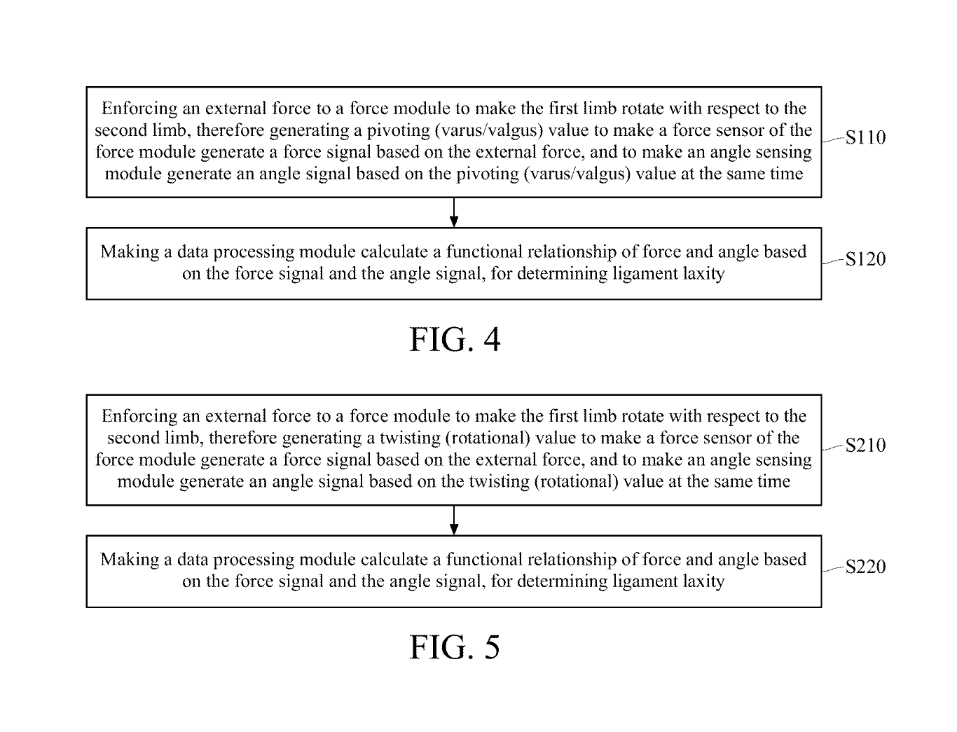

Now the measuring method of the ligament laxity measuring system 10 will be illustrated. The method for measuring the pivoting (varus/valgus) value of the first limb 20 and the second limb 30 will be explained first. FIG. 4 is a flow chart of the process of the measuring method according to the first embodiment of the disclosure. As seen in FIG. 4, an angle between the first limb 20 and the second limb 30 is maintained before measuring. For example, an object may be placed below the second limb 30 to make the first limb 20 or the second limb 30 slightly bent relative to the other. Specifically, the angle is an obtuse angle so the first limb 20 and the horizontal surface form an angle ranging from 25 degrees to 30 degrees, approximately. Then, the force module 100 is installed on the first limb 20 while the fixing module 500 is installed on the second limb 30. Subsequently, the opposite two segments of the angle sensing module 200 (SG150) for measuring the pivoting (varus/valgus) value are attached to the first limb 20 and the second limb 30.

Then, in step S110, an external force is applied on the force module 100 so that the first limb 20 pivots relative to the second limb 30 and therefore generates a pivoting (varus/valgus) value. Thereby, the force sensor 120 generates the force signal based on the external force while the angle sensing module 200 generates the angle signal based on the pivoting (varus/valgus) value. Specifically, a measuring staff put one hand on the fixing handle 520 of the fixing module 500 to fix the relative position of the second limb 30, while putting the other hand on the forcing handle 130 to provide the external force F1. Moreover, the pivoting (varus/valgus) value refers to the angle that the first limb 20 pivots (along the direction of arrow a) with respect to the second limb 30 (thigh) around a pivoting axis L1, activated by the external force F1 applying on the force handle 130 which is located on the lateral side of the first limb 20 (calf) along the X axis (as shown in FIG. 1A). Additionally, the pivoting (varus/valgus) value can be used to determine the degree of laxity in terms of the rotation angle of the anterior cruciate ligament, the posterior cruciate ligament and the lateral cruciate ligaments.

In step S120, the data processing module 300 is made to calculate the functional relationship of force and angle based on the force signal and the angle signal, in order to determine ligament laxity.

Since it is possible to measure the force signal and the angle signal in one measuring procedure, these two signals are measured on the same basis, thereby improving the precision of the ligament laxity measuring system 10. Also, the angle between the first limb 20 and the second limb 30 remain unchanged so the subjects need not to change their positions.

Subsequently, the subject remain the same position and the measuring staff remove the angle sensing module 200 (SG150) and attach the opposite two segments of the angle sensing module 200 (Q150), which is for measuring the twisting (rotational) value, to the first limb 20 and the second limb 30 respectively. This way, the twisting (rotational) value between the first limb 20 and the second limb 30 can be measured.

FIG. 5 is a flow chart of the process of the measuring method according to the second embodiment of the disclosure. As seen in FIG. 5, in step S210, an external force is applied on the force module 100 so that the first limb 20 twists relative to the second limb 30 and therefore generates a twisting (rotational) value. Thereby, the force sensor 120 generates the force signal based on the external force while the angle sensing module 200 generates the angle signal based on the twisting (rotational) value. Specifically, a measuring staff put one hand on the fixing handle 520 of the fixing module 500 to fix the relative position of the second limb 30, while putting the other hand on the forcing handle 130 to provide the external force F2. Thereby, the first limb 20 twists with respect to the second limb 30 to generate a twisting (rotational) value. Additionally, the twisting (rotational) value refers to the angle that the first limb 20 twists (along the direction of arrow b) with respect to the second limb 30 (thigh) around a twisting axis L2, activated by the external force F2 applying on the force handle 130 which is located on the front side of the first limb 20 (calf) along the X axis or by the external force F3 applying on the force handle 130 which is located on the lateral side of the first limb 20 (calf) along the Z axis (as shown in FIG. 1B). The pivoting axis L1 and the twisting axis L2 intersect. In addition, the pivoting axis L1 is parallel to the Z axis while the twisting axis L2 is parallel to the Y axis. Further, the twisting (rotational) value can be used to determine the degree of the laxity regarding the anterior cruciate ligament, the posterior cruciate ligament and the lateral cruciate ligaments.

Then, in step S220, the data processing module 300 is made to calculate the functional relationship of force and angle based on the force signal and the angle signal, in order to determine ligament laxity.

However, the order of measuring the pivoting (varus/valgus) value and the twisting (rotational) value does not intended to limit the disclosure.

In the ligament laxity measuring system and the method for measuring ligament laxity of the disclosure, the ligament laxity measuring system comprises the force sensor of the force module and the angle sensing module which are able to measure the force signal and the angle signal based on the same basis at the same time. As a result, the functional relationship between the force and the angle can be calculated more precisely and this therefore improves precision of the ligament laxity measuring system.

Moreover, it only requires the change of types of angle sensing modules to switch the measurements of the pivoting (varus/valgus) value and the twisting (rotational) value. Thus, this simplifies the measuring processes of the ligament laxity measuring system.

Additionally, the force module is sleeved on the first limb by the straps and the fasteners, which is different from the traditional equipments which involve measuring sizes of the legs to manufacture the corresponding jigs. Thus, the force module of this embodiment is capable of matching the first limbs of different sizes. This reduces the manufacturing cost of the ligament laxity measuring system.

What is claimed is:

1. A ligament laxity measuring system configured for measuring ligament laxity between a first limb and a second limb connected to each other, comprising:
a force module comprising a force sensor, wherein the force module is configured for being disposed on the first limb and for providing an external force to make the first limb pivot or twist with respect to the second limb, thereby generating a pivoting (varus/valgus) value and a twisting (rotational) value, and the force module generates a force signal based on the external force;
an angle sensing module, wherein opposite two segments of the angle sensing module are disposed on the first limb and the second limb respectively, the angle sensing module is configured for sensing the pivoting (varus/valgus) value and the twisting (rotational) value of the first limb and generating an angle signal; and
a data processing module connected to the force sensor of the force module and the angle sensing module, wherein the data processing module is configured for calculating a functional relationship of force and angle based on the force signal and the angle signal, and the ligament laxity is measured based on the functional relationship of force and angle.

2. The ligament laxity measuring system according to claim 1, wherein the force module comprises a first strap and a forcing handle, the first strap is for being detachably sleeved on the first limb, the force sensor is disposed on the first strap and the forcing handle is connected to the force sensor.

3. The ligament laxity measuring system according to claim 1, wherein the force module further comprises a first fastener and a second fastener respectively disposed on opposite two ends of the first strap, the first fastener is detachably fastened with the second fastener such that the first strap is detachably sleeved on the first limb.

4. The ligament laxity measuring system according to claim 1, further comprising a fixing module detachably disposed on the second limb.

5. The ligament laxity measuring system according to claim 4, wherein the fixing module comprises a second strap and a fixing handle, the second strap is detachably sleeved on the second limb and the fixing handle is connected to the second strap.

6. The ligament laxity measuring system according to claim 5, wherein the fixing module further comprises a third fastener and a fourth fastener which are disposed on opposite two ends of the second strap respectively, the third fastener is detachably fastened with the fourth fastener to make the second strap be detachably sleeved on the second limb.

7. The ligament laxity measuring system according to claim 1, wherein the angle sensing module is a strain gauge.

8. The ligament laxity measuring system according to claim 1, wherein the force sensor is a load cell.

9. The ligament laxity measuring system according to claim 1, further comprising a data collection module connected between the force sensor and the data processing module and between the angle sensing module and the data processing module, wherein the data collection module is configured for collecting the force signal and the angle signal.

10. The ligament laxity measuring system according to claim 1, wherein the first limb rotates with respect to the second limb around a pivoting axis and twists with respect to the second limb around a twisting axis, and the pivoting axis and the twisting axis intersect.

11. A method for measuring ligament laxity, configured to measure ligament laxity between a first limb and a second limb connected to each other, comprising the steps of:
enforcing an external force to a force module to make the first limb rotate with respect to the second limb, therefore generating a pivoting (varus/valgus) value to make a force sensor of the force module generate a force signal based on the external force, and to make an angle sensing module generate an angle signal based on the pivoting (varus/valgus) value at the same time; and
making a data processing module calculate a functional relationship of force and angle based on the force signal and the angle signal, for determining ligament laxity.

12. The method for measuring ligament laxity according to claim 11, further comprising: maintaining an angle between the first limb and the second limb, before enforcing the external force to the force module.

13. A method for measuring ligament laxity, configured to measure ligament laxity between a first limb and a second limb connected to each other, comprising the steps of:
enforcing an external force to a force module to make the first limb rotate with respect to the second limb, therefore generating a twisting (rotational) value to make a force sensor of the force module generate a force signal based on the external force, and to make an angle sensing module generate an angle signal based on the twisting (rotational) value at the same time; and
making a data processing module calculate a functional relationship of force and angle based on the force signal and the angle signal, for determining ligament laxity.

14. The method for measuring ligament laxity according to claim 13, further comprising: maintaining an angle between the first limb and the second limb, before enforcing the external force to the force module.
